(19) United States
(12) Patent Application Publication
(10) Pub. No.: US 2026/0041403 A1
(43) Pub. Date: Feb. 12, 2026

(54) IMAGE PROCESSING DEVICE, MEASUREMENT SYSTEM, IMAGE PROCESSING METHOD, AND PROGRAM

(71) Applicant: The University of Electro-Communications, Tokyo (JP)

(72) Inventors: Norihiro KOIZUMI, Chofu-shi (JP); Ryosuke TSUMURA, Chofu-shi (JP); Kodai OKUZAKI, Chofu-shi (JP)

(21) Appl. No.: 19/365,717

(22) Filed: Oct. 22, 2025

Related U.S. Application Data
(63) Continuation of application No. PCT/JP2024/015256, filed on Apr. 17, 2024.

(30) Foreign Application Priority Data
Apr. 26, 2023 (JP) 2023-072467

(51) Int. Cl. A61B 8/00; G06T 7/00

(52) U.S. Cl. CPC A61B 8/5207; A61B 8/5276; A61B 8/543; G06T 7/0014; G06T 2207/10132; G06T 2207/20072; G06T 2207/20081; G06T 2207/30004

(57) ABSTRACT

An image processing device includes a first acquisition unit configured to acquire a first distance image which is a distance image indicating a distance between an imaging target imaged by an imaging device and the imaging device and which is a distance image captured using a part of an animal of which the distance varies with breathing as the imaging target, a second acquisition unit configured to acquire a second distance image which is a distance image captured by the imaging device and which is a distance image captured at a time point different from a time point at which the first distance image has been captured, a difference image generating unit configured to generate a difference image indicating a difference between the acquired first distance image and the acquired second distance image, and a feature part detecting unit configured to detect a feature part.

### TECHNICAL FIELD

The present invention relates to an image processing device, a measurement system, an image processing method, and a program.

This application is a bypass continuation of PCT/JP2024/015256, filed Apr. 17, 2024 which priority is claimed on Japanese Patent Application No. 2023-072467, filed Apr. 26, 2023, the content of which is incorporated herein by reference.

### BACKGROUND ART

In the related art, pressing an ultrasonic probe against an organism and acquiring an ultrasonic image of a region of interest in the organism are performed. There is a technique of tracking a region of interest by changing the position, the pressing angle, or the like of an ultrasonic probe when the region of interest of which an ultrasonic image is acquired moves periodically in the organism (for example, see Patent Document 1). There is a system that causes ultrasonic waves to converge on the tracked region of interest and cauterizes an affected region using the technique described in Patent Document 1. This treatment method is known as high intensity focused ultrasound (HIFU) treatment and is being mainly used to treat a prostate cancer.

### Citation List

### Patent Document

Patent Document 1: Japanese Unexamined Patent Application, First Publication No. 2016-158890

### SUMMARY OF INVENTION

### Technical Problem

Here, when an ultrasonic probe is pressed against an organism, it is first necessary to observe the organism from the outside and to predict a position at which a region of interest is present in the organism. An expert operator can predict the position at which the ultrasonic probe is to be pressed on the basis of appearance features of the organism. However, when it is intended to automatically perform a series of processes of searching for a region of interest on the basis of an ultrasonic image acquired by pressing an ultrasonic probe against an organism and tracking the searched-for region of interest, it is necessary to predict the position at which the ultrasonic probe is to be pressed without employing an expert operator. However, it is not easy to predict a position at which the ultrasonic probe is to be pressed. That is, according to the related art, there is a problem in that it is difficult to estimate a position at which an ultrasonic probe is to be pressed against an organism in order to acquire an ultrasonic image of a region of interest.

The present invention was made in consideration of the aforementioned circumstances, and an objective thereof is to provide an image processing device, a measurement system, an image processing method, and a program that can estimate the position at which an ultrasonic probe is to be pressed against an organism in order to acquire an ultrasonic image of a region of interest.

### Solution to Problem

(1) According to an aspect of the present invention, there is provided an image processing device including a first acquisition unit configured to acquire a first distance image which is a distance image indicating a distance between an imaging target imaged by an imaging device and the imaging device and which is a distance image captured using a part of an animal of which the distance varies with breathing as the imaging target, a second acquisition unit configured to acquire a second distance image which is a distance image captured by the imaging device and which is a distance image captured at a time point different from a time point at which the first distance image has been captured, a difference image generating unit configured to generate a difference image indicating the difference between the acquired first distance image and the acquired second distance image, and a feature part detecting unit configured to detect a feature part which is a boundary part between a varying part and a non-varying part with breathing of the animal which is the imaging target on the basis of the generated difference image.
(2) In the image processing device according to the aspect of (1), the feature part detecting unit may detect the feature part on the basis of a shape of a boundary between a part with a difference and a part without a difference based on the difference image.
(3) In the image processing device according to the aspect of (1) or (2), the feature part detecting unit may include a trained model which has been trained through supervised learning using the difference image and a shape of the feature part determined to correspond to the difference image as training data, and the trained model may estimate the feature part on the basis of the difference image generated by the difference image generating unit.
(4) The image processing device according to the aspect of any one of (1) to (3) may further include a noise reducing unit configured to reduce noise in the difference image by decreasing a difference at a position at which the difference is equal to or greater than a predetermined value in the difference image and a detection unit configured to detect the feature part on the basis of the difference image in which noise has been reduced.
(5) The image processing device according to the aspect of (4) further includes a histogram smoothing unit configured to smooth a histogram based on values of coordinates in the difference image in which noise has been reduced.
(6) According to another aspect of the present invention, there is provided a measurement system including the image processing device according to the aspect of any one of (1) to (5) and a control device that controls a drive device such that the probe moves to a position based on the detected feature part.
(7) According to another aspect of the present invention, there is provided an image processing method including a first acquisition step of acquiring a first distance image which is a distance image indicating a distance between an imaging target imaged by an imaging device and the imaging device and which is a distance image captured using a part of an animal of which the distance varies with breathing as the imaging target, a second acquisition step of acquiring a second distance image which is a distance image captured by the imaging device and which is a distance image captured at a time point different from a time point at which the first distance image has been captured, a difference image generating step of generating a difference image indicating a difference between the acquired first distance image and the acquired second distance image, and a feature part detecting step of detecting a feature part which is a boundary part between a varying part and a non-varying part with breathing of the animal which is the imaging target on the basis of the generated difference image.
(8) According to another aspect of the present invention, there is provided a program causing a computer to perform a first acquisition step of acquiring a first distance image which is a distance image indicating the distance between an imaging target imaged by an imaging device and the imaging device and which is a distance image captured using a part of an animal of which the distance varies with breathing as the imaging target, a second acquisition step of acquiring a second distance image which is a distance image captured by the imaging device and which is a distance image captured at a time point different from a time point at which the first distance image has been captured, a difference image generating step of generating a difference image indicating the difference between the acquired first distance image and the acquired second distance image, and a feature part detecting step of detecting a feature part which is a boundary part between a varying part and a non-varying part with breathing of the animal which is the imaging target on the basis of the generated difference image.

### Advantageous Effects of Invention

According to the present invention, it is possible to estimate a position at which an ultrasonic probe is to be pressed against an organism in order to acquire an ultrasonic image of a region of interest.

### BRIEF DESCRIPTION OF DRAWINGS

FIG. 1 A diagram schematically illustrating a measurement system according to an embodiment.

FIG. 2 A diagram illustrating methods of detecting a region to be measured and acquiring an ultrasonic image which are performed by the measurement system according to the embodiment.

FIG. 3 A functional configuration diagram illustrating an example of a functional configuration of an image processing device according to the embodiment.

FIG. 4 A functional configuration diagram illustrating an example of a functional configuration of a feature part detecting unit according to the embodiment.

FIG. 5 A diagram illustrating change in distance value with breathing acquired by an imaging device according to the embodiment.

FIG. 6 A diagram illustrating an example of a depth image processed by the image processing device according to the embodiment.

FIG. 7 A diagram illustrating an example of an object detecting process that is performed by the image processing device according to the embodiment.

FIG. 8 A diagram illustrating an example of a direction in which scanning with an ultrasonic probe is performed on the basis of a result of the object detecting process performed by the image processing device according to the embodiment.

FIG. 9 A block diagram illustrating an example of an internal configuration of the image processing device according to the embodiment.

### DESCRIPTION OF EMBODIMENTS

### Embodiment

Hereinafter, an exemplary embodiment of an image processing device, a measurement system, an image processing method, and a program according to an aspect of the present invention will be described in detail with reference to the accompanying drawings. Any aspect of the present invention is not limited to such an embodiment and includes various modifications or improvements thereof. That is, elements described below include elements that can be easily thought out by those skilled in the art or elements that are substantially the same. The elements described below can be appropriately combined. Various omissions, substitutions, or modifications of elements can be carried out without departing from the gist of the present invention. In the drawings used for the following description, scales, numbers, and the like of constituent members may be made to be different from actual scales, numbers, and the like of the constituent members in order to make the constituent members be easily recognized.

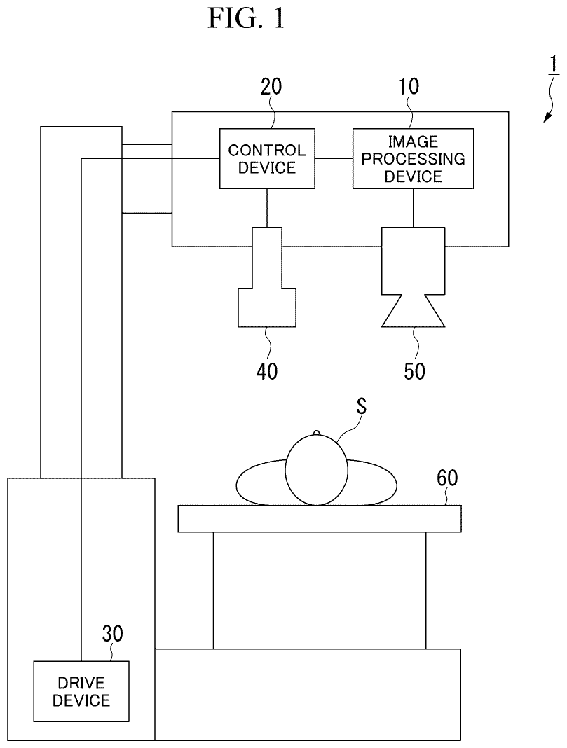

FIG. 1 is a diagram schematically illustrating a measurement system according to an embodiment. The outline of a measurement system 1 will be first described below with reference to the drawing. The measurement system 1 includes an image processing device 10, a control device 20, a drive device 30, a measuring probe 40, an imaging device 50, and an examination table 60. The measurement system 1 includes these devices and thus measures a specific region (for example, an organ) of a subject S. In the following description, a region of the subject S which is a region to be measured by the measurement system 1 may be referred to as a region of interest. Measurement performed by the measurement system 1 may be specifically imaging a region of interest using a predetermined method. Measurement performed by the measurement system 1 may be more specifically fluoroscopic X-ray imaging, computed tomography (CT), magnetic resonance imaging (MRI), single-photon emission computed tomography (SPECT)-CT, tomo-synthesis, or the like. In the following description, it is assumed that the measurement system 1 performs an ultrasonic examination. After measurement has been performed by the measurement system 1, a predetermined surgical procedure based on the result of measurement may be carried out.

In the following description, it is assumed that the subject S which is to be measured by the measurement system 1 is a human being. However, the present embodiment is not limited to this example, and the subject S may be an animal other than a human being.

The subject S is placed on the examination table 60 in a state in which a side of the subject S to be measured of a location to be measured (for example, an organ such as a liver) faces the measuring probe 40 and the imaging device 50. The subject S is placed on the examination table 60 in a state in which the subject faces upward. By placing the subject S facing upward on the examination table 60, it is possible to measure a region of interest from the front side of the subject S. The examination table 60 is driven by the drive device 30 to change the position or the angle in a horizontal direction and a vertical direction.

The drive device 30 changes a relative relationship between the subject S and the measuring probe 40 and the imaging device 50 by changing the position or the angle in the horizontal direction and the vertical direction of the examination table 60. The drive device 30 may change positions or angles in the horizontal direction and the vertical direction of the measuring probe 40 and the imaging device 50 instead of or in addition to changing the position or the angle of the examination table 60.

The imaging device 50 acquires an RGB image (a visible-light image) and depth information (which may also be referred to as a distance value or a depth value) corresponding to each pixel of the RGB image. That is, the imaging device 50 may be an RGBD camera. In the following description, an image including depth information corresponding to coordinates on a two-dimensional plane may be referred to as a distance image. The imaging device 50 acquires an RGB image and a distance image by imaging the subject S.

The image processing device 10 acquires an RGB image and a distance image from the imaging device 50 and performs a predetermined process on the basis of the acquired information. Specifically, the image processing device 10 acquires RGB images and distance images captured at two different time points from the imaging device 50. More specifically, the different time points may be an expiration time and an inspiration time of the subject S. The different time points may be a normal breathing time and a deep breathing time of the subject S. A chest or an abdomen of a human being decreases in size by allowing air to go out of the lungs at the time of expiration and increases in size by allowing air to come into the lungs at the time of inspiration. The size varies depending on an amount of inspiration and an amount of expiration. Accordingly, when the imaging device 50 images the subject S lying to face upward from above in the vertical direction, a distance between the imaging device 50 and the subject S varies between expiration and inspiration. The image processing device 10 estimates a region to be measured of the subject S on the basis of a difference between the distance at the time of expiration and the distance at the time of inspiration. Two different time points at which the imaging device 50 performs imaging are not limited to the expiration time and the inspiration time and may be any times in a range in which a difference appears in the distance image. In the following description, for example, it is assumed that the image processing device 10 performs processing on the basis of RGB images and distance images captured at two different time points of expiration and inspiration.

The control device 20 outputs a control signal for the drive device 30 and the measuring probe 40 on the basis of the region estimated by the image processing device 10. The control device 20 controls a relative relationship between the measuring probe 40 and the subject S by outputting the control signal.

The measuring probe 40 is controlled in a positional relationship with the subject S by the control device 20 and is pressed against the subject S. The measuring probe 40 detects a signal to be measured from the subject S. Specifically, when the measurement system 1 performs an ultrasonic examination, the measuring probe 40 acquires an ultrasonic image from the subject S. The measuring probe 40 outputs the acquired ultrasonic image to the control device 20. In the following description, the measuring probe 40 may be simply referred to as a probe.

The drive device 30 includes a drive mechanism which is not illustrated. The drive mechanism includes components such as a motor, a gear, a pinion, and a rack. The drive device 30 controls a positional relationship between the subject S and the measuring probe 40 and a positional relationship between the subject S and the imaging device 50. The drive device 30 moves the measuring probe 40 to a position at which the subject S is to be measured by controlling the positional relationships.

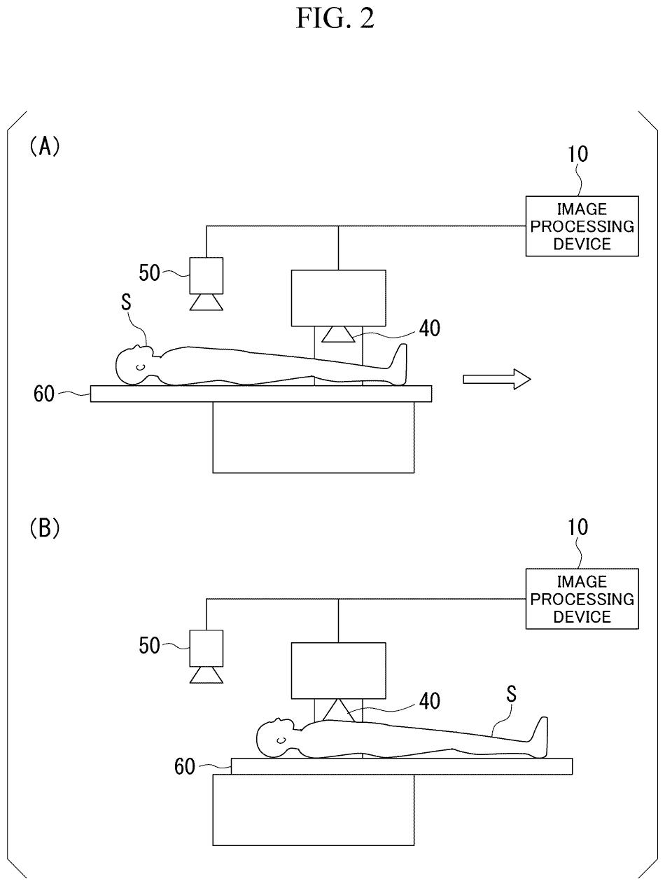

FIG. 2 is a diagram illustrating methods of detecting a region to be measured and acquiring an ultrasonic image which are performed by the measurement system according to the embodiment. The methods of detecting a region to be measured and acquiring an ultrasonic image which are performed by the measurement system 1 will be described below with reference to the drawing. In the following description, it is assumed that the measurement system 1 detects a rib part of the subject S who is a human being and acquires an ultrasonic image of the liver by acquiring an ultrasonic image of a position based on the rib part.

FIG. 2(A) is a diagram of an image for detecting a region to be measured. As illustrated in the drawing, the examination table 60 moves to a position at which a rib of the subject S is included in the angle of view of the imaging device 50. The control device 20 may analyze a region of the subject S to be imaged at a time point at which an RGB image is captured, for example, by analyzing the RGB image captured by the imaging device 50, estimate a substantial position of the rib on the basis of a result of analysis, and move the examination table 60 to a position at which the estimated position is included in the angle of view of the imaging device 50. The imaging device 50 acquires a distance image at the time of expiration of the subject S and a distance image at the time of inspiration at the illustrated position (the position at which the rib of the subject S is included in the angle of view of the imaging device 50). For example, the imaging device 50 may consecutively capture a plurality of distance images and select a distance image at the time of expiration of the subject S and a distance image at the time of inspiration on the basis of the plurality of captured distance images.

Then, the image processing device 10 determines a detailed position of the rib on the basis of the acquired distance image at the time of expiration and the acquired distance image at the time of inspiration. The position of the rib determined by the image processing device 10 may be two-dimensional information or three-dimensional information. The image processing device 10 outputs the determined information on the position of the rib to the control device 20. The control device 20 controls a positional relationship between the subject S and the measuring probe 40 on the basis of the acquired information on the position of the rib and presses the measuring probe 40 against the subject S. The measuring probe 40 acquires an ultrasonic image of the liver from the subject S.

FIG. 2(B) is a diagram of an image for acquiring an ultrasonic image. That is, the drawing illustrates a state in which the measuring probe 40 is pressed against the subject S. In this way, with the measurement system 1, the measuring probe 40 can be pressed to a position at which a region of interest (for example, a liver) of the subject S can be measured without employing the hands of an expert operator.

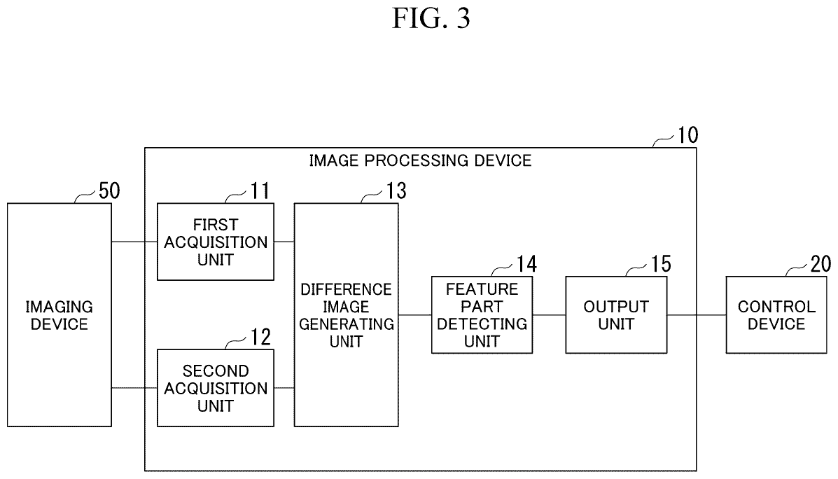

FIG. 3 is a functional configuration diagram illustrating an example of a functional configuration of the image processing device according to the embodiment. An example of the functional configuration of the image processing device 10 will be described below with reference to the drawing. The image processing device 10 includes a first acquisition unit 11, a second acquisition unit 12, a difference image generating unit 13, a feature part detecting unit 14, and an output unit 15. These functional units are realized, for example, using electronic circuits. These functional units may include a storage device such as a semiconductor memory or a magnetic hard disk device therein according to necessity. The functions may be realized by a computer including a central processing unit (CPU) and software.

The first acquisition unit 11 acquires a distance image from the imaging device 50. An imaging target of the imaging device 50 is the subject S. The distance image indicates a distance between the subject S and the imaging device 50. The distance image includes distance values corresponding to coordinates in a two-dimensional plane. The first acquisition unit 11 images a part of an animal in which the distance varies with breathing. A part of an animal in which the distance varies with breathing includes, for example, a chest or an abdomen of a human being. For example, the first acquisition unit 11 acquires a distance image by imaging the chest or the abdomen of a human being. In the following description, the distance image captured by the first acquisition unit 11 may be referred to as a first distance image. The first acquisition unit 11 outputs the acquired first distance image to the difference image generating unit 13.

The second acquisition unit 12 acquires a distance image from the imaging device 50. The second acquisition unit 12 acquires a distance image captured at a time point different from the time point at which the first distance image has been captured. In the following description, the distance image captured by the second acquisition unit 12 may be referred to as a second distance image. The first distance image may be, for example, a distance image acquired at the time of expiration of the subject S, and the second distance image may be, for example, a distance image acquired at the time of inspiration of the subject S. The second acquisition unit 12 outputs the acquired second distance image to the difference image generating unit 13.

The difference image generating unit 13 generates a difference image on the basis of a first distance image acquired by the first acquisition unit 11 and a second distance image acquired by the second acquisition unit 12. The difference image indicates a difference between a distance value of the first distance image and a distance value of the second distance image at each of coordinates in a two-dimensional plane. The values at the coordinates of the difference image can also be said to be differences between the distance values of the first distance image and the distance values of the second distance image. The difference image generating unit 13 outputs the generated difference image to the feature part detecting unit 14.

The feature part detecting unit 14 acquires the difference image generated by the difference image generating unit 13. The feature part detecting unit 14 detects a feature part on the basis of the acquired difference image. The feature part may be, for example, a boundary part between a part varying with breathing of the subject S and a part not varying with breathing. More specifically, the feature part detecting unit 14 may detect a feature part on the basis of a shape of a boundary part between a part with a difference and a part with no difference. A specific example of the feature part is a rib region (a costal arch) of a human being. The feature part detecting unit 14 outputs information for identifying a position of the detected feature part to the output unit 15.

A feature part detecting process performed by the feature part detecting unit 14 may employ a machine learning algorithm or may not employ a machine learning algorithm. An example of the feature part detecting process performed without using the machine learning algorithm is an example using image processing. In the following description, an example of the feature part detecting process using image processing will be described.

First, the feature part detecting unit 14 performs a smoothing process by applying a Gaussian filter or the like to the difference image. The difference image may be a difference image after a noise reducing process and a histogram smoothing process which will be described later. After the smoothing process has been performed, the feature part detecting unit 14 performs a binarization process of the difference image. The binarization process may use a specific threshold value or may use an appropriate threshold value using a technique such as Otsu's binarization. Further, the process of extracting an outline part from the difference image on which the binarization process has been performed which is performed by the feature part detecting unit 14 is also referred to as an outline extracting process. Here, the extracted outline part is nonlinear. Accordingly, the feature part detecting unit 14 approximates the nonlinear outline part using a linear approximate expression. That is, the feature part detecting unit 14 can also be said to approximate the outline part using a plurality of linear functions after the outline extracting process has been performed. This process is also referred to as an outline approximating process. The feature part detecting unit 14 detects a feature part from an angle at a crossing of the plurality of linear functions for approximation. The feature part detecting unit 14 may detect the feature part using information on the length of a line segment or the like in addition to angle information.

The output unit 15 outputs information for identifying a position of the feature part detected by the feature part detecting unit 14 to the control device 20. The information output from the output unit 15 may be for two-dimensionally identifying the feature part or for three-dimensionally identifying the feature part. When the feature part is a rib region (a costal arch) of a human being, the information for identifying the position of the feature part may be coordinate information for identifying an infrasternal angle.

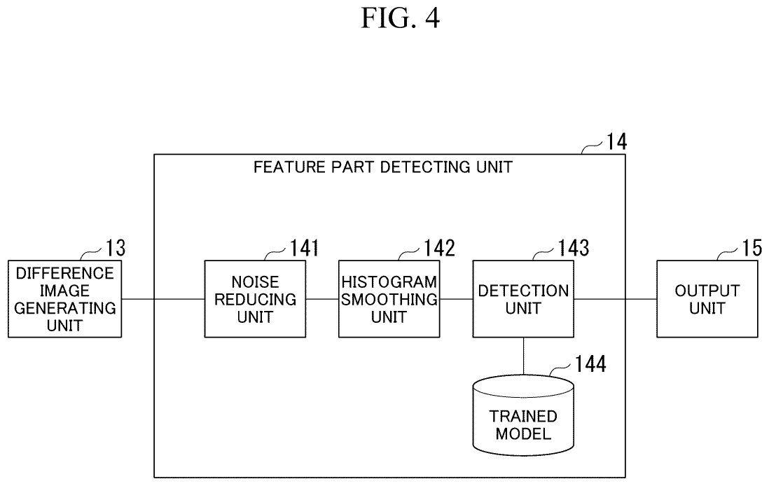

FIG. 4 is a functional configuration diagram illustrating an example of a functional configuration of the feature part detecting unit according to the embodiment. An example of the functional configuration of the feature part detecting unit 14 will be described below with reference to the drawing. The feature part detecting unit 14 includes a noise reducing unit 141, a histogram smoothing unit 142, a detection unit 143, and a trained model 144.

The noise reducing unit 141 acquires a difference image from the difference image generating unit 13. The noise reducing unit 141 performs a noise reducing process on the basis of the acquired difference image. The noise reducing process is a process of reducing noise in the difference image by decreasing the difference at a position at which the difference is equal to or greater than a predetermined value. By performing the noise reducing process, a difference based on breathing remains in the difference image. More specifically, the noise reducing process may be a process of ignoring a value at which the difference is equal to or greater than 150 [mm] and replacing the distance value at the corresponding coordinate with 0 (complementing the distance value with 0).

Here, in the difference image on which the noise reducing process has been performed, the difference may be slight. When the difference is slight, it may be difficult to detect a feature part. Accordingly, the feature part detecting unit 14 performs a difference emphasizing process (that is, a process of enlarging reference scales indicating the difference) such that it is possible to easily (accurately) detect a feature part. This process is also referred to as a histogram smoothing process.

The histogram smoothing unit 142 acquires the difference image on which the noise reducing process has been performed by the noise reducing unit 141. The histogram smoothing unit 142 performs a histogram smoothing process on the difference image. In other words, the histogram smoothing unit 142 can also be said to smooth a histogram based on values at the coordinates of the difference image in which noise has been reduced.

The detection unit 143 acquires the difference image on which the noise reducing process has been performed by the noise reducing unit 141 and the histogram smoothing process has been performed by the histogram smoothing unit 142. The detection unit 143 detects the feature part on the basis of the acquired difference image. A machine learning algorithm may be used for the feature part detecting process performed by the detection unit 143. The trained model 144 which is a learning model trained in advance may be used to perform machine learning.

The trained model 144 is a trained model trained in advance through supervised learning. A difference image and data corresponding to a shape of the feature part determined to correspond to the difference image are used as training data. The difference image used as the training data is preferably a difference image on which the noise reducing process has been performed by the noise reducing unit 141 and the histogram smoothing process has been performed by the histogram smoothing unit 142. The training data for learning may employ a prepared person. The trained model 144 trained in advance estimates a feature part on the basis of an instruction from the detection unit 143.

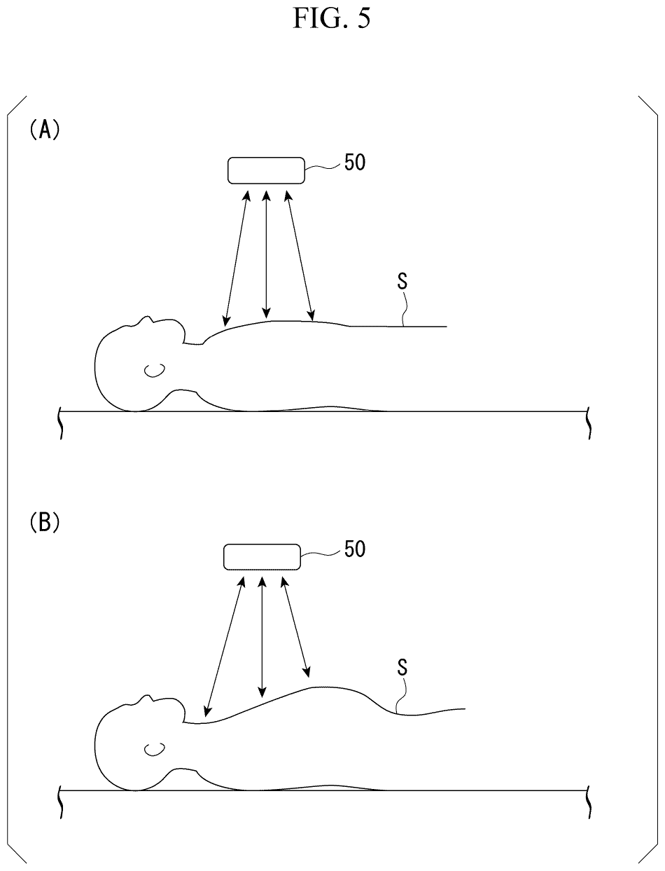

FIG. 5 is a diagram illustrating a change with breathing of a distance value acquired by the imaging device according to the embodiment. The change with breathing of the distance value acquired by the imaging device 50 will be described below with reference to the drawing. FIG. 5(A) illustrates depth information at the time of normal breathing (at a stable inspiration position). In this drawing, the depth (a distance value) acquired by the imaging device 50 is indicated by an arrow. FIG. 5(B) illustrates depth information at the time of deep breathing (at a maximum inspiration position) of the subject S. Similarly, in this drawing, the depth (a distance value) acquired by the imaging device 50 is indicated by an arrow. The time point in the state illustrated in FIG. 5(A) and the time point in the state illustrated in FIG. 5(B) are different time points. As can be seen from comparison between FIG. 5(A) and FIG. 5(B), the depth (the distance value) acquired by the imaging device 50 changes with breathing. The image processing device 10 detects the feature part on the basis of this change in depth (distance value).

In a specific example, the depth information at the time of normal breathing (at a stable inspiration position) illustrated in FIG. 5(A) may be acquired at a normal time (that is, in a state in which an instruction associated with breathing of the subject S is not received). The depth information at the time of deep breathing (at a maximum inspiration position) of the subject S illustrated in FIG. 5(B) may be acquired when a predetermined time (for example, 1 sec) has elapsed after an instruction “take a deep breath” has been given by a speech output unit (or an operator of the measurement system 1) which is not illustrated.

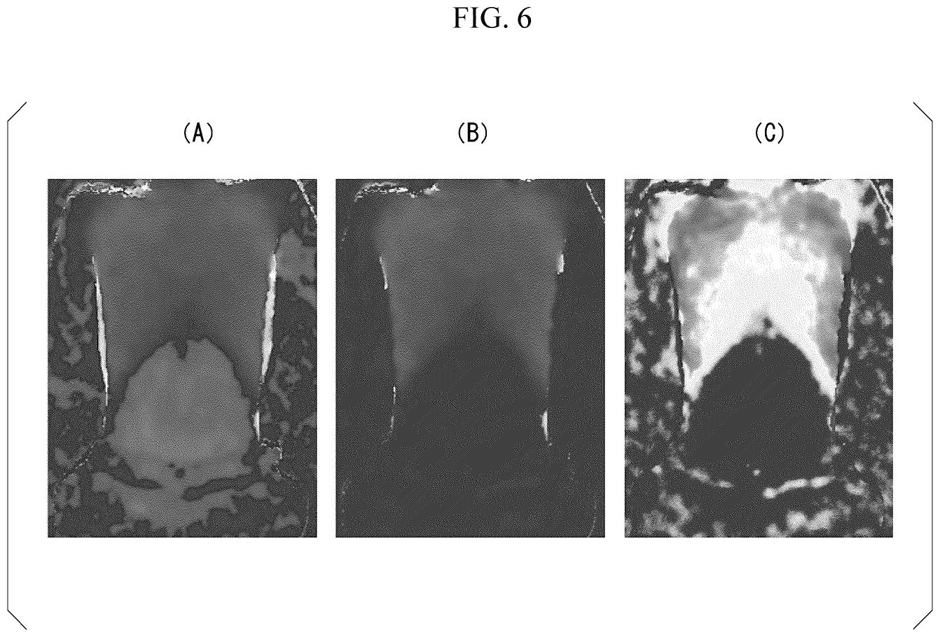

FIG. 6 is a diagram illustrating an example of a depth image processed by the image processing device according to the embodiment. An example of a difference image and an example of a difference image on which the noise reducing process and the histogram smoothing process have been performed will be described below with reference to the drawing. The drawing illustrates an example in which an upper half (a part below a neck) of the subject S is imaged by the imaging device 50. In the illustrated example, the difference images are illustrated in the format of a heat map. However, the difference images according to the present embodiment are not limited to this example. The difference images may have a format other than the format of a heat map.

FIG. 6(A) illustrates an example of a difference image indicating a difference between a first distance image and a second distance image. As illustrated in the drawing, the difference image before the noise reducing process has been performed includes a difference at a plurality of positions. The difference image including distance values having noise components in this way is unclear and is difficult to grasp.

FIG. 6(B) illustrates a difference image indicating an example in which the noise reducing process has been performed on the difference image illustrated in FIG. 6(A). Specifically, in the illustrated example, a process of complementing a position at which the difference is equal to or greater than 150 [mm] with 0 in order to reduce noise is performed. It can be seen that the position at which a difference is generated with breathing becomes clear through the noise reducing process. However, since the difference based on breathing is slight, it can be seen that there occurs a side effect that it is not easy to determine a correct position of a feature part directly from the corresponding image.

FIG. 6(C) illustrates a difference image indicating an example in which the histogram smoothing process has been performed on the difference image illustrated in FIG. 6(B). By performing the histogram smoothing process, it can be seen that the feature part (a costal arch) in the image illustrated in FIG. 6(C) is clearer than the image illustrated in FIG. 6(B). The detection unit 143 according to the present embodiment can more correctly detect the feature part by detecting the feature part on the basis of the difference image processed in this way.

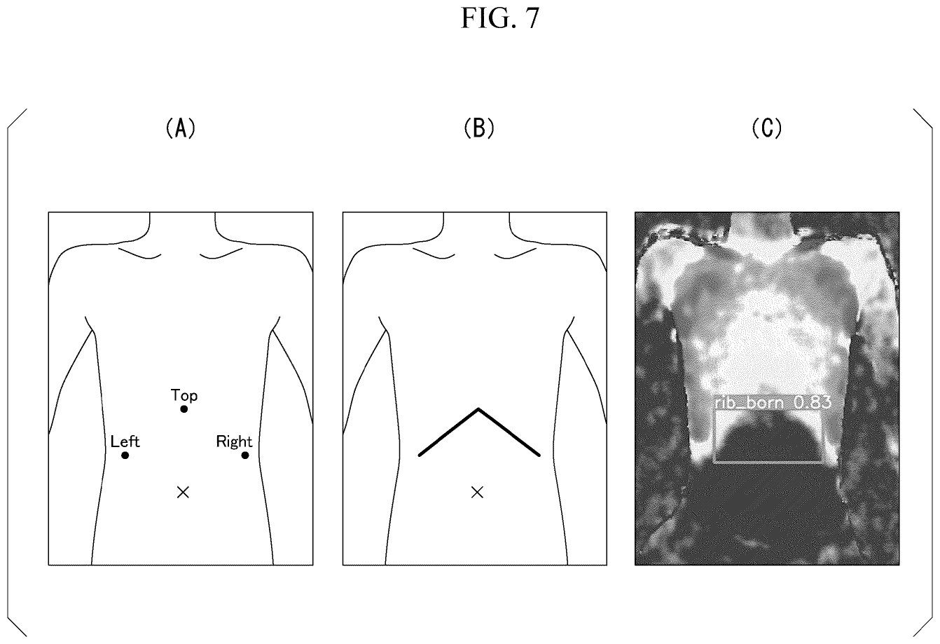

FIG. 7 is a diagram illustrating an example of an object detecting process which is performed by the image processing device according to the embodiment. An example of the object detecting process that is performed by the image processing device 10 will be described below with reference to the drawing. The examples illustrated in FIGS. 7(A) to 7(C) are different examples, and the object detecting process performed by the image processing device 10 can be performed using any method. The illustrated examples are simple examples for realizing the embodiment, and the object detecting process according to the present embodiment is not limited to the illustrated examples.

FIG. 7(A) illustrates an example in which coordinates of three points for identifying a feature part (a costal arch) are detected. As illustrated in the drawing, coordinates of three points “left,” “top,” and “right” are detected. The coordinates of the three points identify a position, a shape, and the like of the costal arch which is a feature part. The coordinates of the three points may be two-dimensional coordinates or three-dimensionally coordinates.

FIG. 7(B) illustrates an example in which line segments for identifying a feature part (a costal arch) are detected. As illustrated in the drawing, two line segments are detected. The coordinates of the two line segments identify a position, a shape, and the like of the costal arch which is a feature part. These line segments may be in two-dimensional plane or a three-dimensional space.

FIG. 7(C) illustrates an example in which a bounding box for identifying a feature part (a costal arch) is detected. As illustrated in the drawing, a bounding box surrounding a feature part is detected. A detected class (rib_born in the illustrated example) and a likelihood of the class (0.83 in the illustrated example) are displayed in an upper part of the bounding box. This bounding box identifies a position, a shape, and the like of the costal arch which is a feature part.

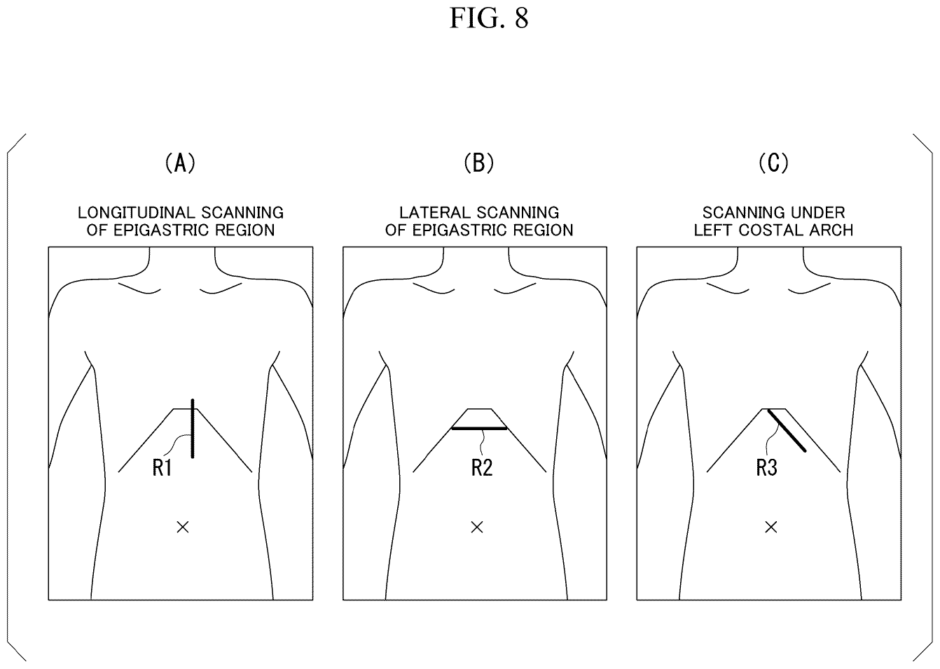

FIG. 8 is a diagram illustrating an example of a direction in which scanning with an ultrasonic probe is performed on the basis of a result of the object detecting process performed by the image processing device according to the embodiment. An example in which the subject S is scanned with the measuring probe 40 by the control device 20 on the basis of the detected feature part (the costal arch) will be described below with reference to the drawing.

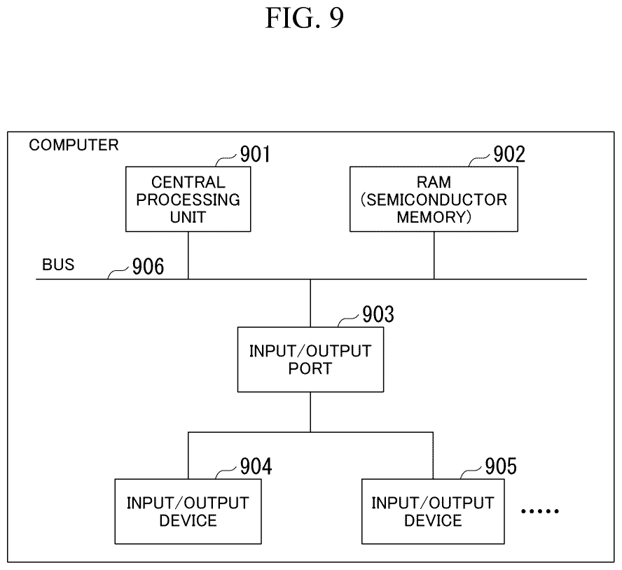

FIG. 9(A) illustrates an example in which longitudinal scanning of an epigastric region is performed. With the longitudinal scanning of an epigastric region, the upper body of the subject S is scanned along the upper body with the measuring probe 40 with respect to the position of the costal arch which is a feature part. FIG. 9(B) illustrates an example in which lateral scanning of an epigastric region is performed. With the lateral scanning of an epigastric region, the upper body of the subject S is scanned in a direction perpendicular to the upper body with the measuring probe 40 with respect to the position of the costal arch which is a feature part. FIG. 9(C) illustrates an example in which scanning under a left costal arch is performed. With the scanning of a left costal arch, scanning with the measuring probe 40 is performed along one line segment of the costal arch with respect to the position of the costal arch which is a feature part. The scanning direction of the measuring probe 40 is not limited to the illustrated examples. Scanning with the measuring probe 40 can be performed in various directions with respect to the detected feature part.

FIG. 9 is a block diagram illustrating an example of an internal configuration of the image processing device according to the embodiment. At least some functions of the image processing device 10 can be realized using a computer. As illustrated in the drawing, the computer includes a central processing unit 901, a RAM 902, an input/output port 903, input/output devices 904 and 905, and a bus 906. The computer itself can be realized using known techniques. The central processing unit 901 executes instructions included in a program read from the RAM 902 or the like. The central processing unit 901 writes data to the RAM 902, read data from the RAM 902, or performs an arithmetic operation or a logical operation in accordance with the instructions. The RAM 902 stores data or programs. Each element included in the RAM 902 has an address and can be accessed using the address. RAM is an abbreviation of “random access memory.” The input/output port 903 is a port for allowing the central processing unit 901 to exchange data with an external input/output device. The input/output devices 904 and 905 are input/output devices. The input/output devices 904 and 905 exchange data with the central processing unit 901 via the input/output port 903. The bus 906 is a shared communication passage which is used in the computer. For example, the central processing unit 901 reads or writes data from or to the RAM 902 via the bus 906. For example, the central processing unit 901 accesses the input/output port via the bus 906.

### Conclusion of Embodiment

According to the aforementioned embodiment, the image processing device 10 includes the first acquisition unit 11 to acquire a first distance image which is a distance image indicating a distance between an imaging target (a subject S) imaged by the imaging device 50 and the imaging device 50 and which is a distance image captured using a part of an animal of which the distance varies with breathing as the imaging target. The image processing device 10 includes the second acquisition unit 12 to acquire a second distance image which is a distance image captured by the imaging device 50 and which is a distance image captured at a time point different from a time point at which the first distance image has been captured. The image processing device 10 includes the difference image generating unit 13 to generate a difference image indicating the difference between the acquired first distance image and the acquired second distance image. The image processing device 10 includes the feature part detecting unit 14 to detect a feature part which is a boundary part between a varying part and a non-varying part with breathing of the animal which is the imaging target on the basis of the generated difference image. The image processing device 10 has this configuration and thus can overcome a problem in that it is difficult to estimate a feature part in the related art and detect a position of a feature part.

With the image processing device 10, since a position of a feature part can be detected, it is possible to scan a position to be measured with the measuring probe 40 by pressing the measuring probe 40 against the position of the feature part. Accordingly, according to the present embodiment, it is possible to perform measurement by pressing the measuring probe 40 against a position to be measured without employing an expert operator. As a result, according to the present embodiment, it is possible to fully automatically perform an examination such as an ultrasonic examination.

According to the present embodiment, the feature part detecting unit 14 detects the feature part on the basis of a shape of a boundary between a part with a difference and a part without a difference based on the difference image generated by the difference image generating unit 13. Accordingly, according to the present embodiment, it is possible to easily detect a feature part on the basis of a distance image.

According to the present embodiment, the feature part detecting unit 14 includes a trained model 144 which has been trained through supervised learning using the difference image and a shape of the feature part determined to correspond to the difference image as training data. The trained model 144 estimates the feature part on the basis of the difference image generated by the difference image generating unit 13. That is, according to the present embodiment, it is possible to detect a feature part using machine learning. As a result, according to the present embodiment, it is possible to accurately detect a feature part.

According to the present embodiment, the feature part detecting unit 14 includes the noise reducing unit 141 to reduce noise in the difference image by decreasing a difference at a position at which the difference is equal to or greater than a predetermined value in the difference image and includes the detection unit 143 to detect the feature part on the basis of the difference image in which noise has been reduced. Here, a raw difference image acquired by simply calculating a difference between the first distance image and the second distance image may include much noise. The noise may be based on the performance of an RGBD sensor. The feature part detecting unit 14 includes the noise reducing unit 141 and thus performs a noise reducing process to remove such noise. Accordingly, according to the present embodiment, since an influence of noise has been reduced, it is possible to accurately detect a feature part.

According to the present embodiment, the image processing device 10 further includes the histogram smoothing unit 142 to perform a histogram smoothing process of smoothing a histogram based on values of coordinates in the difference image in which noise has been reduced. Here, the difference image in which noise has been reduced by the noise reducing unit 141 may have a low resolution indicating a difference and thus may not easily detect a feature part. Therefore, the feature part detecting unit 14 can more accurately detect the difference by further including the histogram smoothing unit 142. As a result, according to the present embodiment, it is possible to accurately detect a feature part.

According to the present embodiment, the measurement system 1 includes measuring probe 40 to detect a signal to be measured from a subject S, includes the drive device 30 to move the measuring probe 40 relative to the subject S, includes the imaging device 50 to capture a first distance image and a second distance image, includes the image processing device 10 to detect a feature part on the basis of the captured first distance image and the captured second distance image, and includes the control device 20 to control the drive device 30 such that the measuring probe 40 moves to a position based on the detected feature part. Accordingly, according to the present embodiment, it is possible to scan the position based on the feature part with the measuring probe 40. As a result, according to the present embodiment, it is possible to perform measurement by pressing the measuring probe 40 against a position to be measured without employing an expert operator. Accordingly, according to the present embodiment, it is possible to fully automatically perform an examination such as an ultrasonic examination.

All or some of the functions of the constituent units provided in the image processing device 10 according to the aforementioned embodiment may be realized by recording programs for realizing these functions on a computer-readable recording medium and causing a computer system to read and execute the programs recorded on the recording medium. The “computer system” mentioned herein includes an OS or hardware such as peripherals.

The “computer-readable recording medium” is a portable medium such as a flexible disk, a magneto-optical disc, a ROM, or a CD-ROM or a storage device such as a hard disk incorporated into a computer system. The “computer-readable recording medium” may include a medium that dynamically holds a program for a short time such as a communication line when the program is transmitted via a network such as the Internet or a communication circuit line such as a telephone line or a medium that holds a program for a predetermined time such as a volatile memory in a computer system serving as a server or a client in that case. The program may be a program for realizing some of the aforementioned functions or may be a program for realizing the aforementioned functions in combination with another program stored in advance in the computer system.

While embodiments of the present invention have been described above with reference to the drawings, any specific configuration is not limited to the embodiments and includes a change in design without departing from the gist of the present invention. The embodiments described above may be independent embodiments, or arbitrary embodiments may be combined.

### INDUSTRIAL APPLICABILITY

According to the present invention, it is possible to estimate a position at which an ultrasonic probe is to be pressed against an organism in order to acquire an ultrasonic image of a region of interest.

### REFERENCE SIGNS LIST

1 . . . Measurement system, 10 . . . Image processing device, 20 . . . Control device, 30 . . . Drive device, 40 . . . Measuring probe, 50 . . . Imaging device, 60 . . . Examination table, S . . . Subject, 11 . . . First acquisition unit, 12 . . . Second acquisition unit, 13 . . . Difference image generating unit, 14 . . . Feature part detecting unit, 15 . . . Output unit, 141 . . . Noise reducing unit, 142 . . . Histogram smoothing unit, 143 . . . Detection unit, 144 . . . Trained model

1. An image processing device comprising:
a first acquisition unit configured to acquire a first distance image which is a distance image indicating a distance between an imaging target imaged by an imaging device and the imaging device and which is a distance image captured using a part of an animal of which the distance varies with breathing as the imaging target;
a second acquisition unit configured to acquire a second distance image which is a distance image captured by the imaging device and which is a distance image captured at a time point different from a time point at which the first distance image has been captured;
a difference image generating unit configured to generate a difference image indicating a difference between the acquired first distance image and the acquired second distance image; and
a feature part detecting unit configured to detect a feature part which is a boundary part between a varying part and a non-varying part with breathing of the animal which is the imaging target on the basis of the generated difference image.

2. The image processing device according to claim 1, wherein the feature part detecting unit detects the feature part on the basis of a shape of a boundary between a part with a difference and a part without a difference based on the difference image.

3. The image processing device according to claim 1, wherein the feature part detecting unit includes a trained model which has been trained through supervised learning using the difference image and a shape of the feature part determined to correspond to the difference image as training data, and
wherein the trained model estimates the feature part on the basis of the difference image generated by the difference image generating unit.

4. The image processing device according to claim 1, further comprising:
a noise reducing unit configured to reduce noise in the difference image by decreasing a difference at a position at which the difference is equal to or greater than a predetermined value in the difference image; and
a detection unit configured to detect the feature part on the basis of the difference image in which noise has been reduced.

5. The image processing device according to claim 4, further comprising a histogram smoothing unit configured to smooth a histogram based on values of coordinates in the difference image in which noise has been reduced.

6. A measurement system comprising:
a probe configured to detect a signal to be measured from an imaging target;
a drive device configured to move the probe relative to an animal which is the imaging target;
an imaging device configured to capture a first distance image and a second distance image;
the image processing device according to claim 1; and
a control device configured to control the drive device such that the probe moves to a position based on the detected feature part.

7. An image processing method comprising:
a first acquisition step of acquiring a first distance image which is a distance image indicating a distance between an imaging target imaged by an imaging device and the imaging device and which is a distance image captured using a part of an animal of which the distance varies with breathing as the imaging target;
a second acquisition step of acquiring a second distance image which is a distance image captured by the imaging device and which is a distance image captured at a time point different from a time point at which the first distance image has been captured;
a difference image generating step of generating a difference image indicating a difference between the acquired first distance image and the acquired second distance image; and
a feature part detecting step of detecting a feature part which is a boundary part between a varying part and a non-varying part with breathing of the animal which is the imaging target on the basis of the generated difference image.

8. A program causing a computer to perform:
a first acquisition step of acquiring a first distance image which is a distance image indicating a distance between an imaging target imaged by an imaging device and the imaging device and which is a distance image captured using a part of an animal of which the distance varies with breathing as the imaging target;
a second acquisition step of acquiring a second distance image which is a distance image captured by the imaging device and which is a distance image captured at a time point different from a time point at which the first distance image has been captured;
a difference image generating step of generating a difference image indicating a difference between the acquired first distance image and the acquired second distance image; and
a feature part detecting step of detecting a feature part which is a boundary part between a varying part and a non-varying part with breathing of the animal which is the imaging target on the basis of the generated difference image.
